(19) United States
(12) Patent Application Publication
(10) Pub. No.: US 2011/0294223 A1
(43) Pub. Date: Dec. 1, 2011

(54) APPARATUS AND METHOD FOR CHARACTERIZING PARAMETERS FOR THE CRACKING, IN-SITU COMBUSTION, AND UPGRADING OF HYDROCARBONS

(76) Inventors: Kambiz Safinya, Houston, TX (US); Anthony Robert Kovscek, San Carlos, CA (US); Bo Chen, Stanford, CA (US)

(21) Appl. No.: 12/791,719

(22) Filed: Jun. 1, 2010

(51) Int. Cl. G01N 25/20; G01N 33/00

(52) U.S. Cl. 436/147; 422/83

(57) ABSTRACT

An apparatus for characterizing parameters for the cracking, in-situ combustion, and upgrading of hydrocarbons includes a reactor defining a chamber, a temperature probe operably associated with the reactor, and a gas inlet in fluid communication with the chamber. The apparatus further comprises a gas outlet in fluid communication with the chamber and an electromagnetic radiation attenuating material configured to heat the reactor when the electromagnetic radiation attenuating material is irradiated by electromagnetic radiation.

### BACKGROUND OF THE INVENTION

1. Field of the Invention

The present invention relates to an apparatus and method for characterizing parameters for the cracking, in-situ combustion, and upgrading of hydrocarbons.

2. Description of Related Art

Hydrocarbon reservoirs contain naturally-occurring hydrocarbon molecules that are extracted by a variety of methods. The method or methods selected depend upon, for example, the quality and composition of the hydrocarbons, as well as the reservoir pressures and temperatures. In some situations, hydrocarbons are heated within the reservoir to enhance production rates of the hydrocarbons and to recover hydrocarbons that are otherwise not typically recoverable. The introduction of heat into the reservoir can also allow conversion of naturally-occurring hydrocarbon molecules into more valuable chemical species, as well as rejecting unwanted elements, such as sulfur and heavy metals, from naturally-occurring hydrocarbon molecules. This process is commonly referred to as “in-situ upgrading.” Hydrocarbons are also upgraded using heat at the surface, after they have been extracted from hydrocarbon reservoirs.

The design, execution, and management of such thermal recovery methods are based upon a continuous series of laboratory measurements of kinetic and transport properties of the chemical reactions of field samples of rock plus hydrocarbon fluid. The results of such measurements are converted into engineering parameters that are used to execute the production strategy and manage the hydrocarbon field. Such a method is depicted in FIG. 1, in which a field sample is collected (block 101) and prepared for testing (block 103). Portions of the prepared sample are processed in a combustion tube (block 105) and in a kinetic cell (block 107). Combustion tube measurements are made to determine whether the reservoir is suitable for production using a combustion recovery method, the required air injection rates to sustain combustion, and combustion temperatures. Measurements are made in the kinetic cell to determine reaction rates, reaction products, and by-products as a function of temperature. Data from the combustion tube and kinetic cell processing steps are converted into engineering parameters (block 109), which are then communicated to the field (block 111). The engineering parameters are used in the field to manage operations (block 113). The method is repeated as needed or desired.

Such conventional measurements are made using stainless steel reactors disposed within electric furnaces. Set up, heating, and cool down of such conventional equipment is time consuming. Accordingly, about one week is required to collect the desired data for one field sample using conventional techniques, which hinders throughput.

There are devices and methods for characterizing the kinetic and transport properties of the chemical reactions of field samples that are well known in the art; however, considerable shortcomings remain.

### BRIEF SUMMARY OF THE INVENTION

In one aspect, the present invention provides an apparatus for characterizing parameters for the cracking, in-situ combustion, and upgrading of hydrocarbons. The apparatus comprises a reactor defining a chamber, a temperature probe operably associated with the reactor, and a gas inlet in fluid communication with the chamber. The apparatus further comprises a gas outlet in fluid communication with the chamber and an electromagnetic radiation attenuating material configured to heat the reactor when the electromagnetic radiation attenuating material is irradiated by electromagnetic radiation.

In another aspect, the present invention provides a system for characterizing parameters for the cracking, in-situ combustion, and upgrading of hydrocarbons. The system includes an apparatus comprising a reactor defining a chamber, a temperature probe operably associated with the reactor, and a gas inlet in fluid communication with the chamber. The apparatus further comprises a gas outlet in fluid communication with the chamber and an electromagnetic radiation attenuating material configured to heat the reactor when the electromagnetic radiation attenuating material is irradiated by electromagnetic radiation. The system further comprises an electromagnetic radiation source for irradiating the electromagnetic radiation attenuating material.

In yet another aspect, the present invention provides a method for characterizing parameters for the cracking, in-situ combustion, and upgrading of hydrocarbons. The method comprises disposing a sample in a chamber defined by a reactor of an apparatus, the apparatus including an electromagnetic radiation attenuating material configured to heat the reactor when the electromagnetic radiation attenuating material is irradiated by electromagnetic radiation; introducing a gas into the chamber; and irradiating the electromagnetic radiation attenuating material with electromagnetic radiation. The method further comprises monitoring a temperature of the sample and analyzing an effluent emitted from the chamber.

The present invention provides significant advantages, including (1) providing rapid heating rates that simulate heating rates expected in a hydrocarbon reservoir during, for example, in-situ combustion of crude oil, in-situ conversion of oil shale, in-situ coal gasification, and the like; (2) providing a small reactor size and thermal mass that allows rapid quenching for analysis of post-process reactor contents versus the extent of reaction; (3) providing improved sample throughput in analysis operations; (4) providing further improved sample output in analysis operations when the apparatus of the present invention is utilized in serial or parallel modes due to rapid heating and cooling of the samples; (5) providing a reactor that can be heated by either microwave electromagnetic radiation or radio frequency electromagnetic radiation; and (6) providing a readily scalable system and method for characterizing parameters for the cracking, in-situ combustion, and upgrading of hydrocarbons by testing multiple samples, either serially or in parallel.

Additional objectives, features and advantages will be apparent in the written description which follows.

### BRIEF DESCRIPTION OF THE DRAWINGS

The novel features of the invention are set forth in the appended claims. However, the invention itself, as well as a preferred mode of use, and further objectives and advantages thereof, will best be understood by reference to the following detailed description when read in conjunction with the accompanying drawings, in which the leftmost significant digit(s) in the reference numerals denote(s) the first figure in which the respective reference numerals appear, wherein:

FIG. 1 is block flow diagram, illustrating a conventional method for determining kinetic and transport properties of chemical reactions of field samples;

FIG. 2 is a perspective view of a first illustrative embodiment of a system for characterizing parameters for the cracking, in-situ combustion, and upgrading of hydrocarbons;

FIG. 3 is a partial cross-sectional view of an apparatus for use in characterizing parameters for the cracking, in-situ combustion, and upgrading of hydrocarbons of FIG. 2, taken along line 3-3 in FIG. 2;

FIG. 4 is a perspective view of a second illustrative embodiment of a system for characterizing parameters for the cracking, in-situ combustion, and upgrading of hydrocarbons;

FIG. 5 is a partial cross-sectional view of an apparatus for use in characterizing parameters for the cracking, in-situ combustion, and upgrading of hydrocarbons of FIG. 4, taken along line 5-5 in FIG. 4;

FIG. 6 is a perspective view of a third illustrative embodiment of a system for characterizing parameters for the cracking, in-situ combustion, and upgrading of hydrocarbons;

FIG. 7 is a perspective view of a fourth illustrative embodiment of a system for characterizing parameters for the cracking, in-situ combustion, and upgrading of hydrocarbons;

FIG. 8 is a perspective view of a fifth illustrative embodiment of a system for characterizing parameters for the cracking, in-situ combustion, and upgrading of hydrocarbons;

FIG. 9 is a perspective view of a sixth illustrative embodiment of a system for characterizing parameters for the cracking, in-situ combustion, and upgrading of hydrocarbons;

FIG. 10 is a block flow diagram illustrating a method of the present invention for determining kinetic and transport properties of chemical reactions of field samples;

FIG. 11 is a graphical representation of a temperature ramp of a sample as processed using an apparatus of the present invention heated using an electromagnetic radiation source; and

FIG. 12 is a graphical representation of data derived from chromatographic analysis of gases exiting a gas outlet of an apparatus for characterizing parameters for the cracking, in-situ combustion, and upgrading of hydrocarbons.

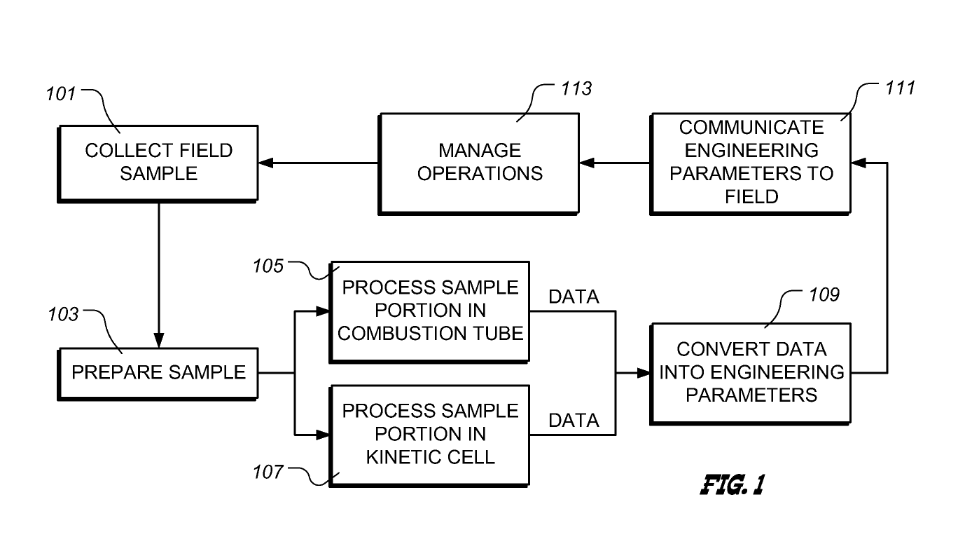

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related, safety-related, and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present invention relates to the cracking, in-situ combustion, and upgrading of hydrocarbons in surface facilities and within hydrocarbon reservoirs, such as, for example, oil, oil shale, and coal reservoirs. Specifically, the present invention relates to an apparatus and method for characterizing parameters for the cracking, in-situ combustion, and upgrading of hydrocarbons. The apparatus and method measure the kinetics of chemical reactions at realistic heating rates using electromagnetic technology within rock samples saturated with hydrocarbons. The apparatus and method are useful in assisting in the design and execution of fossil fuel production and upgrading. The present invention utilizes electromagnetic radiation to obtain rapid, even, and tunable heating, and tunable reactions within samples. While the present invention represents a batch method, it provides rapid sample throughput to accommodate multiple samples in a timely fashion. For example, sample temperature, reaction products, and sample by-products are measured in substantially real time. These measurements are interpreted to reveal chemical reaction kinetics of the hydrocarbons being studied. In one embodiment, the present method can be completed in about a day, as compared to about a week for conventional techniques.

Heating of the sample is achieved using an electromagnetic radiation source, such that electromagnetic radiation is absorbed by the sample of interest and the chamber holding the sample. The radiation source can provide, for example, microwave or radio frequency radiation. The chamber that holds the sample, and/or the sample itself, comprises one or more materials that promote heating of the chamber, chosen based at least upon the frequency of the radiation employed. In one implementation, the sample chamber, or one or more components of the sample chamber, exhibit suitable dielectric constants and loss factors to promote rapid heating for a predetermined microwave frequency. In another implementation, the sample chamber, or one or more components of the sample chamber, exhibit suitable, high electromagnetic permeabilities such that radio frequency radiation generates eddy currents within the sample chamber or the one or more components of the sample chamber. Electrical resistance exhibited by the chamber or the one or more components of the sample chamber results in Joule heating in these members and in the sample of interest. The present system includes a temperature probe disposed within the chamber so that temperature within the chamber can be measured substantially continuously. The sample chamber, or the one or more components of the sample chamber, exhibit significant absorption of electromagnetic radiation at a predetermined frequency to generate heat. Moreover, the sample chamber, or the one or more components of the sample chamber, are generally thermally stable and generally nonreactive when operated within a temperature range of about 25 degrees Celsius to about 1200 degrees Celsius.

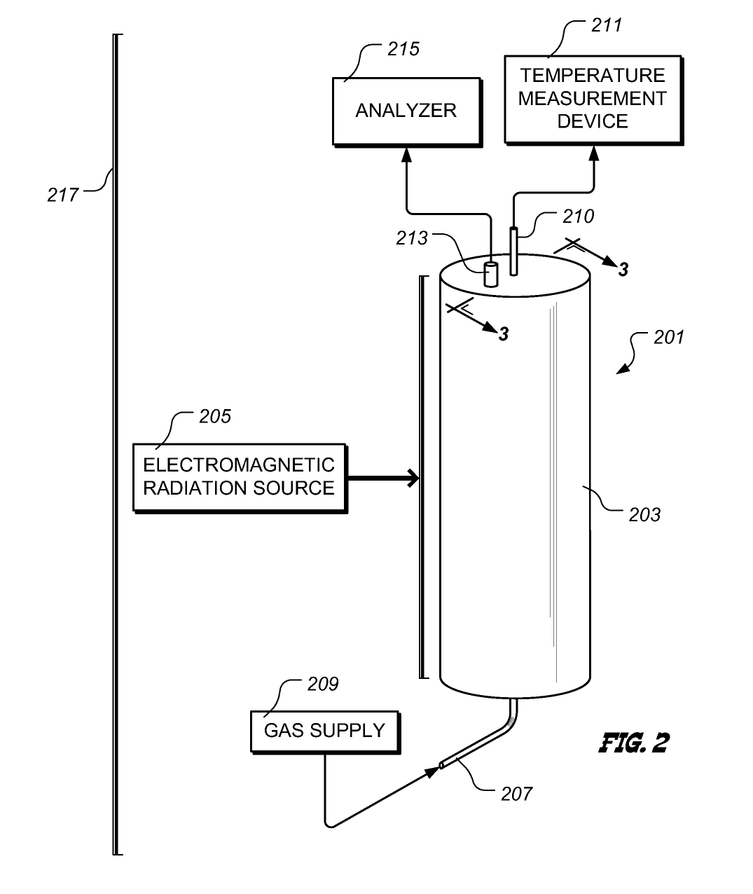

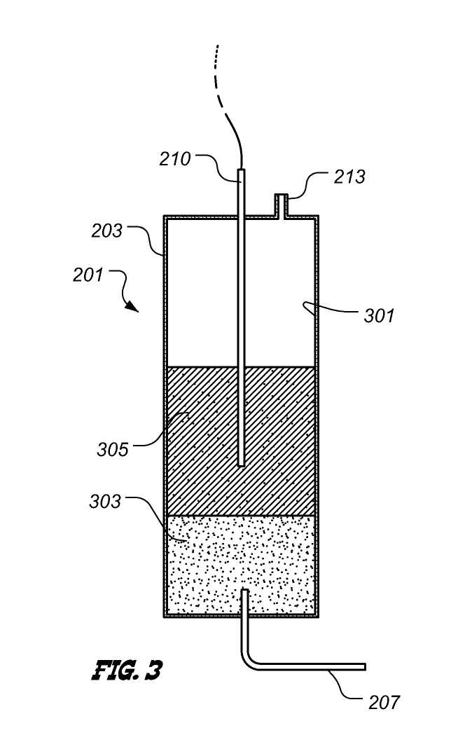

FIG. 2 depicts a perspective view of a first illustrative embodiment of an apparatus 201 for use in characterizing parameters for the cracking, in-situ combustion, and upgrading of hydrocarbons, and further depicts other equipment operably associated with apparatus 201, as is described in further detail herein. FIG. 3 depicts a partial cross-sectional view of apparatus 201, taken along line 3-3 in FIG. 2. Apparatus 201 comprises a reactor 203 defining a sample chamber 301. In the illustrated embodiment, reactor 203 incorporates a material that strongly attenuates electromagnetic radiation. Thus, when reactor 203 is subjected to electromagnetic radiation, such as from an electromagnetic radiation source 205, the temperature of reactor 203 is increased by the effects of the electromagnetic radiation. A gas inlet 207 sealingly extends through reactor 203 and is in fluid communication with sample chamber 301. A gas dispersion medium 303 is disposed within sample chamber 301, such that gas inlet 207 is operably associated with gas dispersion medium 303. In the illustrated embodiment, gas inlet 207 extends into and terminates within gas dispersion medium 303. Gas inlet 207 provides a conduit for one or more gases, such as air, to be introduced from a gas supply 209 into sample chamber 301. When in use, a sample 305, such as a field sample, is disposed within sample chamber 301, such that gas provided via gas inlet 207 flows through gas dispersion medium 303 before encountering sample 305. Gas dispersion medium 303 aids in substantially evenly distributing the flow of gas through sample 305. A temperature probe 210 sealingly extends through reactor 203 into sample chamber 301 and, when in use, into sample 305. Note that temperature probe 210 is not shown in cross-section in FIG. 3. Temperature probe 210 is coupled with a temperature measurement device 211 to monitor temperatures of sample 305. A gas outlet 213 penetrates reactor 203 and is in fluid communication with sample chamber 301. Thus, during operation, gases produced from sample 305, along with the one or more gases introduced into sample chamber 301 via gas inlet 207, whether in their original, introduced form or chemically combined with elements and/or compounds from sample 305, exit sample chamber 301 via gas outlet 213 to an analyzer 215, such as a gas chromatograph. Apparatus 201, electromagnetic radiation source 205, gas supply 209, temperature measurement device 211, and analyzer 215 make up a system 217 for characterizing parameters for the cracking, in-situ combustion, and upgrading of hydrocarbons.

While the embodiment discussed herein concerning FIGS. 2 and 3 provides reactor 203 that incorporates a material that strongly attenuates electromagnetic radiation, the scope of the present invention is not so limited. Rather, a material that strongly attenuates electromagnetic radiation may be interspersed in sample 305 rather than being incorporated into reactor 203. In another embodiment, a material that strongly attenuates electromagnetic radiation is both interspersed in sample 305 and is incorporated into reactor 203.

The present method of characterizing parameters for the cracking, in-situ combustion, and upgrading of hydrocarbons utilizing the embodiment of FIGS. 2 and 3 includes disposing sample 305 in sample chamber 301, such that temperature probe 210 is disposed in sample 305. Gas supply 209 is activated to introduce a gas into sample chamber 301. Electromagnetic radiation source 205 is activated to direct electromagnetic radiation onto reactor 203. It should be noted that the depiction of electromagnetic radiation being directed onto reactor 203 from one direction is merely illustrative for clarity. Preferably, electromagnetic radiation is directed onto reactor 203 from several directions about a circumference of reactor 203. Temperature measurement device 211 is monitored to determine temperatures of sample 305, and electromagnetic radiation source 205 is controlled to maintain a desired temperature or range of temperatures. Gases emitted from gas outlet 213 are analyzed by analyzer 215 to determine the particular makeup of the gases. The present method may be performed by automated means or by human, manual means.

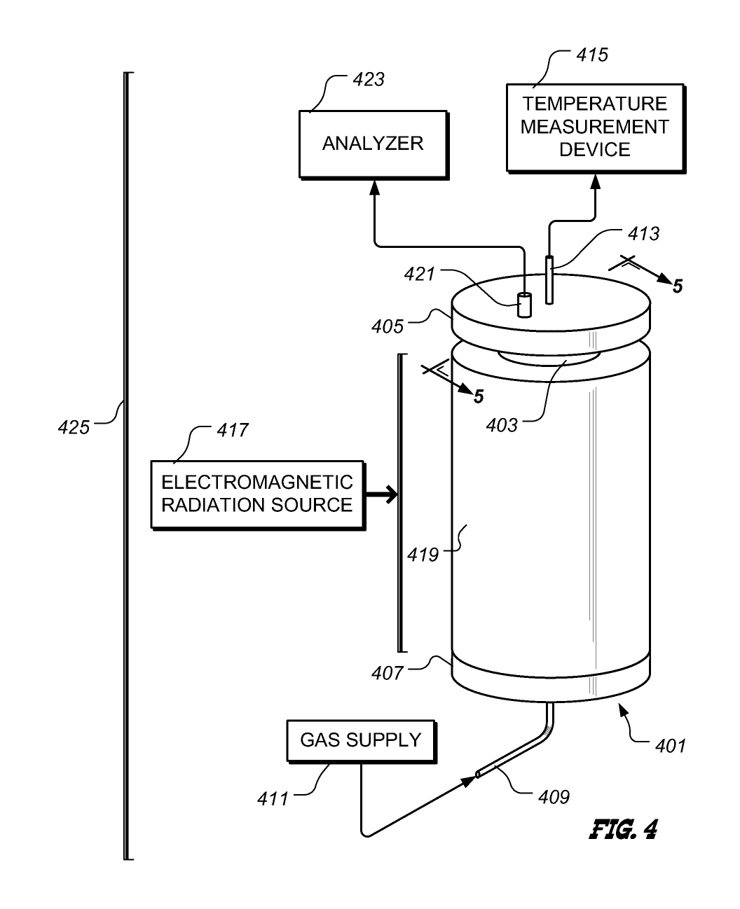

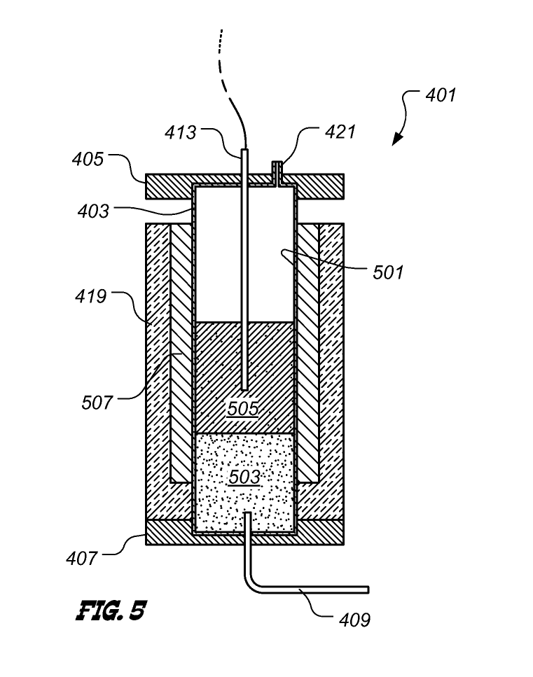

FIG. 4 depicts a perspective view of a second illustrative embodiment of an apparatus 401 for use in characterizing parameters for the cracking, in-situ combustion, and upgrading of hydrocarbons and further depicts other equipment operably associated with apparatus 401, as is described in further detail herein. FIG. 5 depicts a partial cross-sectional view of apparatus 401, taken along line 5-5 in FIG. 4. Apparatus 401 comprises a generally radiolucent reactor 403, made from a material such as, for example, quartz, defining a sample chamber 501. In other words, reactor 403 is generally not affected by electromagnetic radiation. A first tube fitting 405 is disposed at one end of reactor 403 and a second tube fitting 407 is disposed at an opposing end of reactor 403, such that reactor 403 is seated into tube fittings 405 and 407. A gas inlet 409 sealingly extends through second tube fitting 407 and reactor 403 and is in fluid communication with sample chamber 501. A gas dispersion medium 503 is disposed within sample chamber 501, such that gas inlet 409 is operably associated with gas dispersion medium 503. In the illustrated embodiment, gas inlet 409 extends into and terminates within gas dispersion medium 503. Gas inlet 409 provides a conduit for one or more gases, such as air, to be introduced from a gas supply 411 into sample chamber 501. When in use, a sample 505, such as a field sample, is disposed within sample chamber 501, such that gas provided via gas inlet 409 flows through gas dispersion medium 503 before encountering sample 505. Gas dispersion medium 503 aids in substantially evenly distributing the flow of gas through sample 505. A temperature probe 413 sealingly extends through first tube fitting 405 and reactor 403 into sample chamber 501 and, when in use, into sample 505. Note that temperature probe 413 is not shown in cross-section in FIG. 5. Temperature probe 413 is coupled with a temperature measurement device 415 to monitor temperatures of sample 505.

Still referring to FIGS. 4 and 5, an attenuation member 507 is disposed about at least a portion of reactor 403. Attenuation member 507 incorporates a material that strongly attenuates electromagnetic radiation. Thus, when attenuation member 507 is subjected to electromagnetic radiation, such as from an electromagnetic radiation source 417, the temperature of reactor 403 is increased by the effects of the electromagnetic radiation upon attenuation member 507. Disposed about attenuation member 507 and, in some embodiments, at least a portion of reactor 403 revealed from attenuation member 507, is an insulation member 419 to inhibit heat loss from attenuation member 507 and reactor 403. A gas outlet 421 penetrates first tube fitting 405 and reactor 403 and is in fluid communication with sample chamber 501. Thus, during operation, gases produced from sample 505, along with the one or more gases introduced into sample chamber 501 via gas inlet 409, whether in their original, introduced form or chemically combined with elements and/or compounds from sample 505, exit sample chamber 501 via gas outlet 421 to an analyzer 423, such as a gas chromatograph. Apparatus 401, electromagnetic radiation source 417, gas supply 411, temperature measurement device 415, and analyzer 423 make up a system 425 for characterizing parameters for the cracking, in-situ combustion, and upgrading of hydrocarbons.

While the embodiment discussed herein concerning FIGS. 4 and 5 provides attenuation member 507 that incorporates a material that strongly attenuates electromagnetic radiation, the scope of the present invention is not so limited. Rather, a material that strongly attenuates electromagnetic radiation may be interspersed in sample 505 rather than providing attenuation member 507. In another embodiment, a material that strongly attenuates electromagnetic radiation is both interspersed in sample 505 and is incorporated into attenuation member 507.

The present method of characterizing parameters for the cracking, in-situ combustion, and upgrading of hydrocarbons utilizing the embodiment of FIGS. 4 and 5 includes disposing sample 505 in sample chamber 501, such that temperature probe 413 is disposed in sample 505. Gas supply 411 is activated to introduce a gas into sample chamber 501. Electromagnetic radiation source 417 is activated to direct electromagnetic radiation through insulation member 419 onto attenuation member 507. It should be noted that the depiction of electromagnetic radiation being directed onto attenuation member 507 from one direction is merely illustrative for clarity. Preferably, electromagnetic radiation is directed onto attenuation member 507 from several directions about a circumference of attenuation member 507. Temperature measurement device 415 is monitored to determine temperatures of sample 505, and electromagnetic radiation source 417 is controlled to maintain a desired temperature or range of temperatures. Gases emitted from gas outlet 421 are analyzed by analyzer 423 to determine the particular makeup of the gases. The present method may be performed by automated means or by human, manual means.

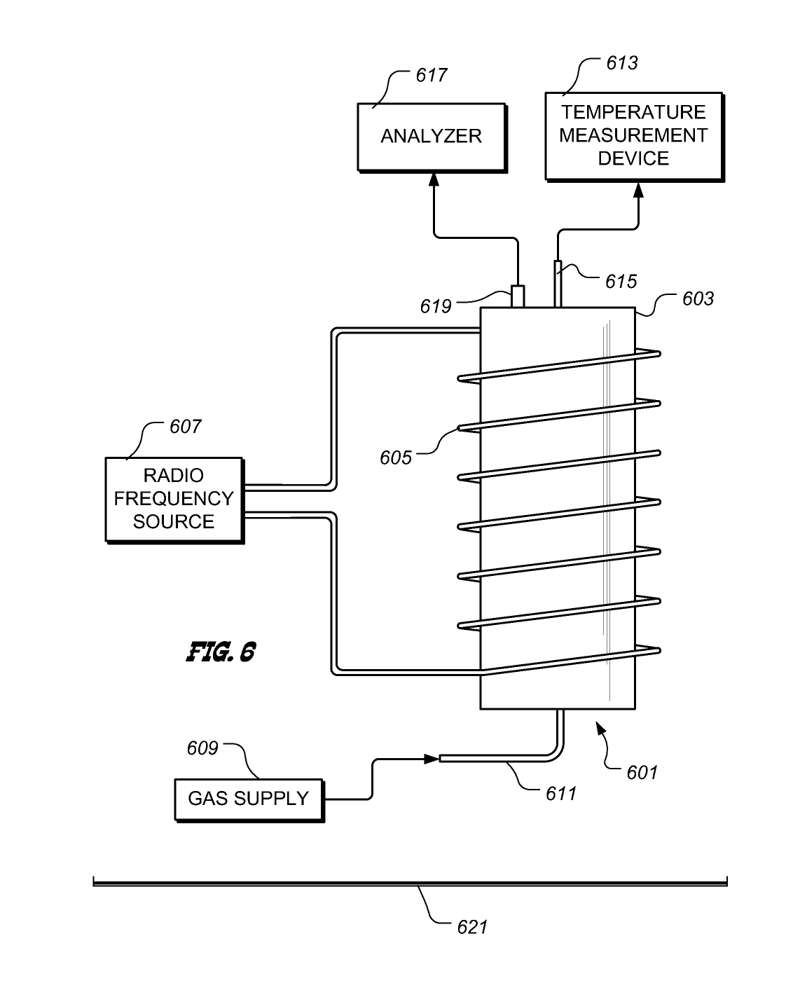

While the embodiments of FIGS. 2-5 can employ electromagnetic radiation sources that provide either microwave or radio frequency radiation to heat a sample, such as samples 305 and 505, for analysis, FIG. 6 depicts a third illustrative embodiment of an apparatus 601 for use in characterizing parameters for the cracking, in-situ combustion, and upgrading of hydrocarbons that employs radio frequency radiation to heat a sample for analysis. FIG. 6 further depicts other equipment operably associated with apparatus 601, as is described in further detail herein. In the illustrated embodiment, apparatus 601 comprises a reactor 603 that incorporates one or more materials that exhibit high electromagnetic permeability, such as iron or the like. An induction coil 605 is disposed about reactor 603 and is coupled with a radio frequency source 607. In operation, radio frequency electrical energy is applied to induction coil 605. Radio frequency electromagnetic radiation is emitted from induction coil 605 to produce eddy currents in reactor 603. The eddy currents generate Joule-type heating in the high electromagnetic permeability material or materials of reactor 603. Reactor 603, in turn, provides induction heating to the sample therein. It should be noted that in some embodiments the material or materials of high electromagnetic permeability may be incorporated into the sample disposed within reactor 603 or the material or materials of high electromagnetic permeability may be incorporated into both reactor 603 and into the sample disposed in reactor 603.

Reactor 603 and the other devices operably associated with reactor 603 and shown in FIG. 6 operate as discussed herein concerning the embodiment of FIGS. 2 and 3. A gas supply 609 provides one or more gases that flow into reactor 603 via a gas inlet 611. A temperature measurement device 613 is coupled with a temperature probe 615 to monitor the temperature of the sample disposed within reactor 603. An analyzer 617, such as a gas chromatograph, analyzes gases that flow from reactor 603 via a gas outlet 619. Other aspects of reactor 603 correspond to those of another embodiment disclosed herein, or their equivalents. Apparatus 601, radio frequency source 607, gas supply 609, temperature measurement device 613, and analyzer 617 make up a system 621 for characterizing parameters for the cracking, in-situ combustion, and upgrading of hydrocarbons.

While sample heating in the embodiments of FIGS. 2-6 is rapid, data analysis throughput is improved with greater measurement density. Accordingly, FIG. 7 depicts an illustrative embodiment of a system 701 for characterizing parameters for the cracking, in-situ combustion, and upgrading of hydrocarbons utilizing a plurality of apparatuses 703 comprising reactors 704 for processing samples. Note that, in FIG. 7, only one apparatus 703 and one reactor 704 are labeled in the interest of clarity. In the illustrated embodiment, each of the plurality of apparatuses 703 corresponds to apparatus 201 of FIGS. 2 and 3; however, the scope of the present invention is not so limited. Rather, one or more of the plurality of apparatuses can take on the form of any embodiment disclosed herein or their equivalents. For example, reactors 704 may include one or more materials that strongly attenuate electromagnetic radiation and/or samples disposed in reactors 704 may incorporate one or more materials that strongly attenuate electromagnetic radiation. System 701 further includes an electromagnetic field generator 705 coupled with a power source 707 for powering electromagnetic field generator 705. Electromagnetic field generator 705 emits an electromagnetic field that operates on the one or more materials that strongly attenuate electromagnetic radiation in each of reactors 704. As in other embodiments disclosed herein, a gas supply 709 provides one or more gases to reactors 704. A temperature measurement device 711 independently monitors temperatures of each sample disposed in the plurality of apparatuses 703 via temperature probes 713 (only one labeled in FIG. 7 in the interest of clarity). An analyzer 715, such as a gas chromatograph, independently analyses gases emitted from each gas outlet 717 (only one labeled in FIG. 7 in the interest of clarity) of the plurality of apparatuses 703. The scope of the present invention, however, contemplates a plurality of temperature measurement devices, such as temperature measurement device 711, and/or a plurality of analyzers, such as analyzer 715, corresponding to the plurality of apparatuses 703, such that one temperature measurement device and/or one analyzer is operatively associated with each apparatus 703.

In one implementation of the embodiment of FIG. 7, different amounts of one or more materials that attenuate electromagnetic radiation are incorporated into each reactor 704 and/or into samples disposed in reactors 704 to obtain different rates of heating from the same electromagnetic radiation source, i.e., electromagnetic field generator 705. In another implementation, generally identical samples are disposed in each reactor 704 with generally identical amounts of attenuation material. Individual reactors 704 are removed at predetermined time intervals from electromagnetic field generator 705, so that the reaction products may be analyzed to understand the extent of reaction and the reaction pathway.

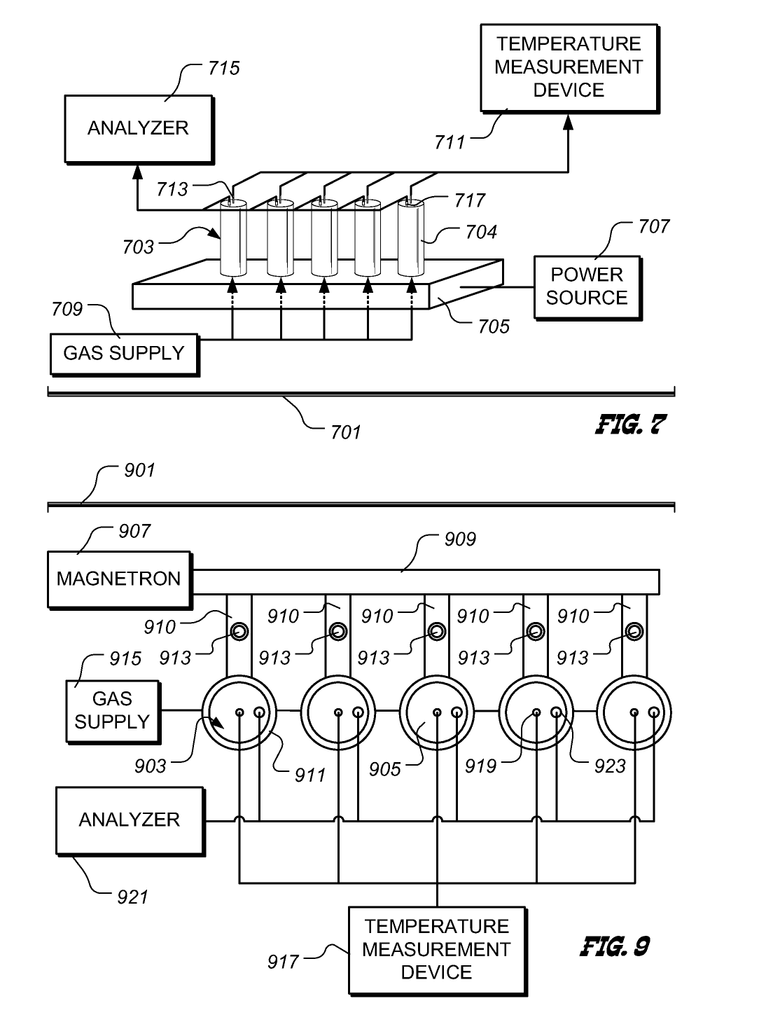

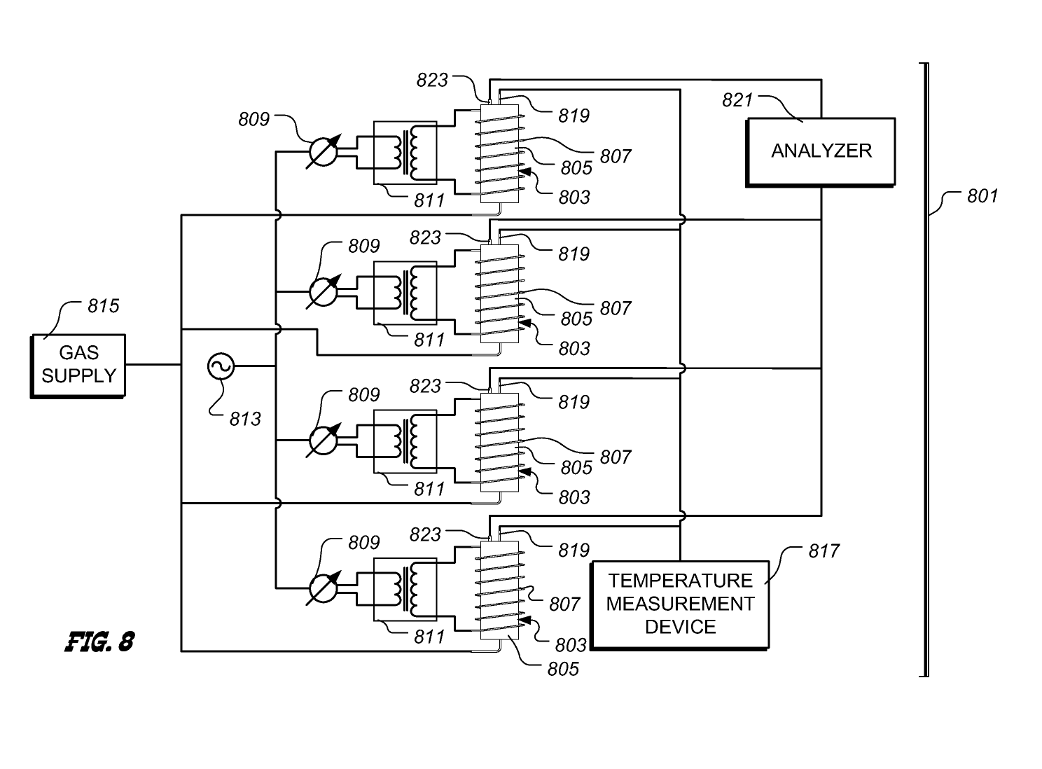

FIG. 8 schematically depicts an alternative, illustrative embodiment of a system 801 that provides greater measurement density, as compared to systems of the present invention that include only one reactor. In the embodiment of FIG. 8, system 801 comprises a plurality of apparatuses 803 comprising reactors 805 for processing samples. In the illustrated embodiment, each of the plurality of apparatuses 803 corresponds to apparatus 601 of FIG. 6; however, the scope of the present invention is not so limited. Rather, one or more of the plurality of apparatuses can take on the form of any embodiment disclosed herein or their equivalents. For example, reactors 805 may include one or more materials that strongly attenuate electromagnetic radiation and/or samples disposed in reactors 805 may incorporate one or more materials that strongly attenuate electromagnetic radiation. System 801 further comprises an induction coil 807 disposed about each reactor 805. A control device 809 tunes the frequency and power input to each induction coil 807, via a transformer 811. A power source 813 provides electrical power to each control device 809 and, ultimately, to each induction coil 807. As in other embodiments disclosed herein, a gas supply 815 provides one or more gases to reactors 805. A temperature measurement device 817 independently monitors temperatures of each sample disposed in the plurality of apparatuses 803 via temperature probes 819. An analyzer 821, such as a gas chromatograph, independently analyses gases emitted from each gas outlet 823 of the plurality of apparatuses 803. The scope of the present invention, however, contemplates a plurality of temperature measurement devices, such as temperature measurement device 817, and/or a plurality of analyzers, such as analyzer 821, corresponding to the plurality of apparatuses 803, such that one temperature measurement device and/or one analyzer is operatively associated with each apparatus 803. In the operation of this embodiment, each sample can be subjected to a different rate of heating.

FIG. 9 depicts another alternative, illustrative embodiment of a system 901 that provides greater measurement density, as compared to systems of the present invention that include only one reactor. In the embodiment of FIG. 9, system 901 comprises a plurality of apparatuses 903 comprising reactors 905 for processing samples. Note that in FIG. 9 only one apparatus 903 and one reactor 905 are labeled in the interest of clarity. In the illustrated embodiment, each of the plurality of apparatuses 903 corresponds to any embodiment disclosed herein, or their equivalents, that are heated using microwave radiation. For example, reactors 905 may include one or more materials that strongly attenuate microwave radiation and/or samples disposed in reactors 905 may incorporate one or more materials that strongly attenuate microwave radiation. System 901 further comprises a magnetron 907 for generating microwave radiation. A main waveguide 909 guides microwave radiation emitted from magnetron 907 to a plurality of waveguide branches 910 leading to a plurality of microwave cavities 911. One apparatus 903 of the plurality of apparatuses 903 is disposed in each microwave cavity 911 of the plurality of microwave cavities 911. An attenuator 913 is operatively associated with each of the plurality of waveguide branches 910 to regulate the amount of microwave radiation reaching each of microwave cavities 911. Thus, system 901 provides precise, tunable heating of each individual sample disposed in reactors 905. As in other embodiments disclosed herein, a gas supply 915 provides one or more gases to reactors 905. A temperature measurement device 917 independently monitors temperatures of each sample disposed in the plurality of apparatuses 903 via temperature probes 919 (only one labeled in the interest of clarity). An analyzer 921 (only one labeled in the interest of clarity), such as a gas chromatograph, independently analyses gases emitted from each gas outlet 923 of the plurality of apparatuses 903. The scope of the present invention, however, contemplates a plurality of temperature measurement devices, such as temperature measurement device 917, and/or a plurality of analyzers, such as analyzer 921, corresponding to the plurality of apparatuses 903, such that one temperature measurement device and/or one analyzer is operatively associated with each apparatus 903.

It should be noted that, in any of the embodiments disclosed herein or their equivalents, a temperature probe, such as temperature probe 210, 413, 615, 713, 819, 919, or the like, can take on the form of any suitable temperature probe. For example, such a temperature probe can take on the form of a shielded thermocouple, a temperature-sensitive optical fiber, or the like.

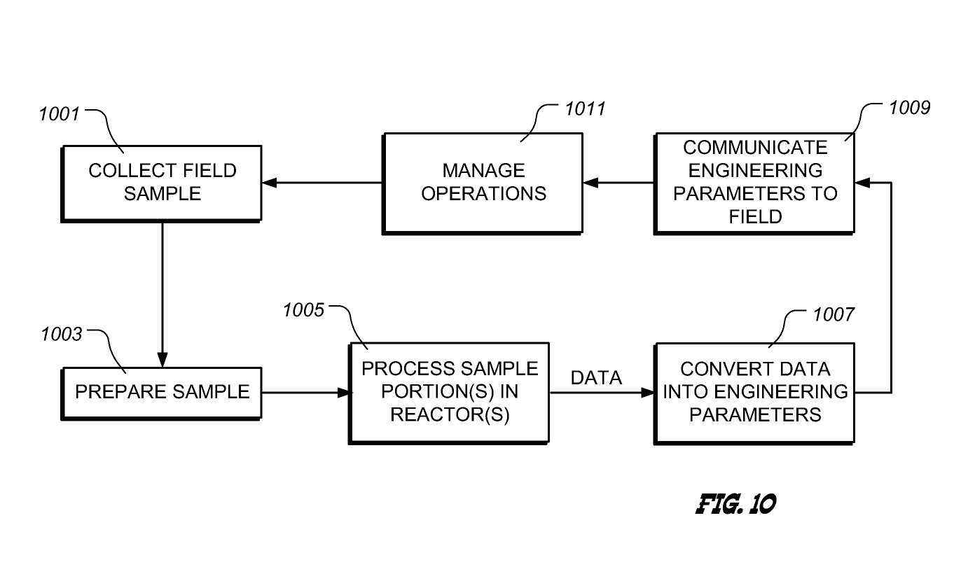

FIG. 10 depicts an illustrative embodiment of a method for characterizing parameters for the cracking, in-situ combustion, and upgrading of hydrocarbons and managing oilfield operations based upon the characterized parameters. In the illustrative embodiment, a field sample is collected (block 1001) and prepared for testing (block 1003). One or more portions of the prepared sample, or portions from a plurality of samples in implementations wherein multiple sample portions are tested, are processed in a reactor of the present invention (block 1005). Data from the reactor processing step are converted into engineering parameters (block 1007), which are then communicated to the field (block 1009). The engineering parameters are used in the field to manage operations (block 1011). The method is repeated as needed or desired.

FIG. 11 depicts a graphical representation of exemplary data corresponding to a temperature ramp of a sample as processed using an apparatus, such as apparatus 201 of FIGS. 2 and 3, and electromagnetic radiation source of the present invention. In this example, the electromagnetic radiation attenuation material is mixed directly into the sample being tested. FIG. 3 shows that heating of the sample is rapid, smooth, and controllable.

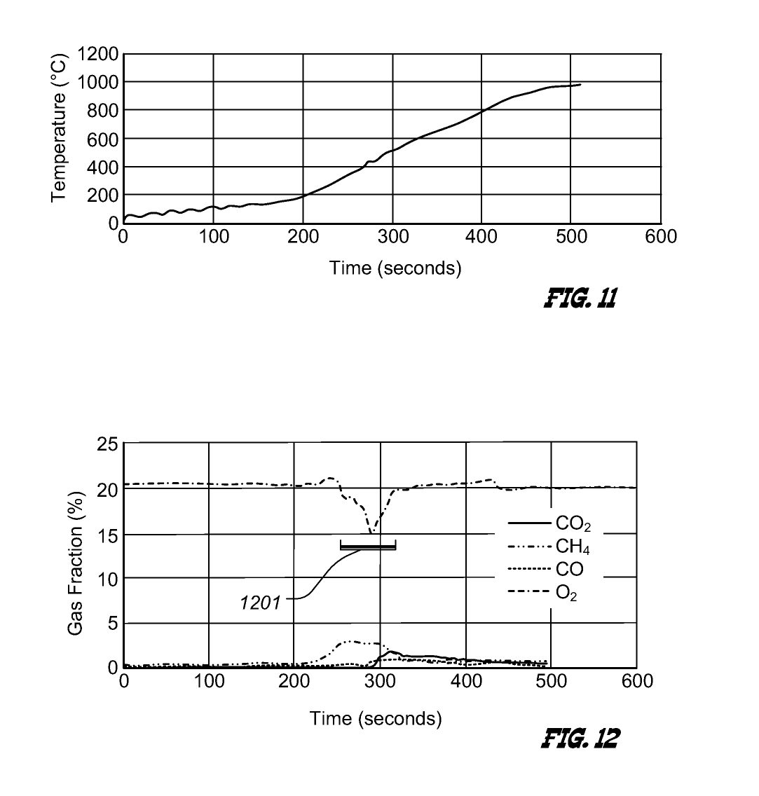

FIG. 12 depicts a graphical representation of exemplary data derived from chromatographic analysis of gases exiting a gas outlet of an apparatus for characterizing parameters for the cracking, in-situ combustion, and upgrading of hydrocarbons, such as apparatus 401 of FIGS. 4 and 5. In this example, the sample disposed in the reactor of the apparatus is a mixture of hydrocarbon, water, and sand. The gas introduced into the reactor is compressed air. Data shown in FIG. 12 for the effluent gas illustrate consumption of oxygen (O₂) during the experiment.

The present invention provides an improved method for the kinetics of oxidation, pyrolysis, and cracking of hydrocarbons to be measured. The data, such as the data represented in FIGS. 11 and 12, can be direct inputs into advanced isoconversional analysis techniques that provide input for field assessment of in-situ combustion, cracking, and pyrolysis of hydrocarbons.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope of the invention. Accordingly, the protection sought herein is as set forth in the claims below. Although the present invention is shown in a limited number of forms, it is not limited to just these forms, but is amenable to various changes and modifications.

What is claimed is:

1. An apparatus for characterizing parameters for the cracking, in-situ combustion, and upgrading of hydrocarbons, comprising:
a reactor defining a chamber;
a temperature probe operably associated with the reactor;
a gas inlet in fluid communication with the chamber;
a gas outlet in fluid communication with the chamber, and
an electromagnetic radiation attenuating material configured to heat the reactor when the electromagnetic radiation attenuating material is irradiated by electromagnetic radiation.

2. The apparatus of claim 1, wherein the reactor defines a wall that includes the electromagnetic radiation attenuating material.

3. The apparatus of claim 1, wherein the apparatus further comprises a sample disposed in the chamber, wherein the sample includes the electromagnetic radiation attenuating material.

4. The apparatus of claim 3, wherein the reactor defines a wall that includes the electromagnetic radiation attenuating material.

5. The apparatus of claim 1, further comprising an attenuation member disposed about the reactor, wherein the attenuation member includes the electromagnetic radiation attenuating material.

6. The apparatus of claim 5, wherein the apparatus further comprises a sample disposed in the chamber, wherein the sample includes the electromagnetic radiation attenuating material.

7. The apparatus of claim 1, further comprising a gas dispersion medium disposed in the chamber, such that the gas inlet terminates in and is in fluid communication with the gas dispersion medium.

8. A system for characterizing parameters for the cracking, in-situ combustion, and upgrading of hydrocarbons, comprising:
an apparatus, comprising:
a reactor defining a chamber;
a temperature probe operably associated with the reactor;
a gas inlet in fluid communication with the chamber;
a gas outlet in fluid communication with the chamber, and
an electromagnetic radiation attenuating material configured to heat the reactor when the electromagnetic radiation attenuating material is irradiated by electromagnetic radiation; and
an electromagnetic radiation source for irradiating the electromagnetic radiation attenuating material.

9. The system of claim 8, wherein the electromagnetic radiation source is a microwave radiation source.

10. The system of claim 8, wherein the electromagnetic radiation source is a radio frequency radiation source.

11. The system of claim 10, wherein the radio frequency radiation source comprises:
a radio frequency source; and
an induction coil coupled with the radio frequency source and disposed about the reactor.

12. The system of claim 8, further comprising a temperature measurement device operatively associated with the temperature probe.

13. The system of claim 8, further comprising a gas supply operatively associated with the gas inlet.

14. The system of claim 8, further comprising an analyzer operatively associated with the gas outlet.

15. The system of claim 14, wherein the analyzer is a gas chromatograph.

16. The system of claim 14, wherein the apparatus is replaced with a plurality of apparatuses, each of the apparatuses comprising:
a reactor defining a chamber;
a temperature probe operably associated with the reactor;
a gas inlet in fluid communication with the chamber;
a gas outlet in fluid communication with the chamber, and
an electromagnetic radiation attenuating material configured to heat the reactor when the electromagnetic radiation attenuating material is irradiated by electromagnetic radiation,
such that the electromagnetic radiation source is configured to radiate electromagnetic radiation upon the electromagnetic radiation attenuating material of each apparatus.

17. The system of claim 16, wherein the electromagnetic radiation source is an electromagnetic field generator.

18. The system of claim 16, wherein the electromagnetic radiation source comprises:
a power source; and
a plurality of induction coils coupled with the power source, wherein an induction coil of the plurality of induction coils is disposed about each reactor of the plurality of apparatuses.

19. The system of claim 18, further comprising a plurality of control devices, wherein a control device of the plurality of control devices is operatively associated with the power source and one of the induction coils of the plurality of induction coils, such that the control device is operable to regulate the amount of electromagnetic radiation emitted from the induction coil.

20. The system of claim 16, wherein the electromagnetic radiation source is a magnetron and the system further comprises:
a plurality of microwave cavities; and
a waveguide extending between the magnetron and the plurality of microwave cavities, wherein one apparatus of the plurality of apparatuses is disposed in each microwave cavity of the plurality of microwave cavities.

21. The system of claim 20, wherein the waveguide comprises:
a main waveguide extending from the magnetron;
a plurality of waveguide branches extending between the main waveguide and each microwave cavity of the plurality of microwave cavities; and
a plurality of control devices, each control device of the plurality of control devices operatively associated with a waveguide branch of the plurality of waveguide branches to regulate the amount of electromagnetic radiation propagated to the microwave chamber to which the waveguide branch extends.

22. A method for characterizing parameters for the cracking, in-situ combustion, and upgrading of hydrocarbons, comprising:
disposing a sample in a chamber defined by a reactor of an apparatus, the apparatus including an electromagnetic radiation attenuating material configured to heat the reactor when the electromagnetic radiation attenuating material is irradiated by electromagnetic radiation;
introducing a gas into the chamber;
irradiating the electromagnetic radiation attenuating material with electromagnetic radiation;
monitoring a temperature of the sample; and
analyzing an effluent emitted from the chamber.

23. The method of claim 22, wherein irradiating the electromagnetic radiation attenuating material with electromagnetic radiation is accomplished by operating a magnetron to emit microwave radiation.

24. The method of claim 22, wherein irradiating the electromagnetic radiation attenuating material with electromagnetic radiation is accomplished by operating an electromagnetic field generator to emit radio frequency radiation.

25. The method of claim 22, further comprising:
converting data generated by analyzing the effluent emitted from the chamber into engineering parameters;
communicating the engineering parameters to the field; and
managing operations in the field based at least upon the engineering parameters.
